(19) United States
(12) Patent Application Publication
(10) Pub. No.: US 2017/0326146 A1
(43) Pub. Date: Nov. 16, 2017

(54) METHODS OF TREATING HEPATITIS B INFECTIONS AND RELATED DOSAGE REGIMES

(71) Applicant: ContraVir Pharmaceuticals, Inc., Edison, NJ (US)

(72) Inventor: John SULLIVAN-BOLYAI, Edison, NJ (US)

(21) Appl. No.: 15/591,340

(22) Filed: May 10, 2017

Related U.S. Application Data
(60) Provisional application No. 62/335,602, filed on May 12, 2016.

(51) Int. Cl. A61K 31/52; A61K 9/00

(52) U.S. Cl. CPC A61K 31/52; A61K 9/0053

(57) ABSTRACT

The present invention relates to compounds and methods for treating viral infections (e.g., Hepatitis B viral infections). The compounds of the disclosure are described by formula I:

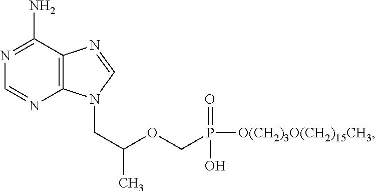

or a pharmaceutically acceptable salt, stereoisomer, a diastereomer, an enantiomer or racemate thereof.

### RELATED APPLICATIONS

This application claims the benefit of and priority to U.S. Provisional Application No. 62/335,602, filed May 12, 2016, the content of which is hereby incorporated by reference in its entirety.

### TECHNICAL FIELD

The present disclosure is directed to treatment of a viral infection (e.g., hepatitis B infection) with a phosphonate ester of tenofovir.

### BACKGROUND

The hepatitis B virus (HBV) afflicts 400 million individuals worldwide and causes an estimated 600,000 deaths each year from complications arising from HBV infection. The primary goal of treating chronic hepatitis B is to suppress HBV replication and induce liver disease remission prior to the onset of cirrhosis and hepatocellular carcinoma, which are often the result of an HBV infection if not managed. The limited efficacy of current antiviral treatments highlights the need for new therapeutic tools for treating chronic HBV. Currently, there are seven FDA-approved drugs for the treatment of chronic HBV, including interferon-alpha, pegylated interferon-alpha, lamivudine, entecavir, telbivudine, adefovir dipivoxil, and tenofovir disporoxil fumarate. No currently approved drug is completely effective at suppressing HBV replication permanently.

3-(hexadecyloxy)propyl hydrogen ((R)-1-(6-amino-9H-purin 9-yl) propan-2-yloxy)methylphosphonate (referred to as CMX157, hexadecyloxypropyl tenofovir or HDP-TFV), a lipid conjugate of tenofovir, was designed to mimic lysophosphatidylcholine to take advantage of natural lipid uptake pathways and to achieve high intracellular concentrations of the active antiviral, with the aim of increasing the effectiveness of tenofovir (TFV) against HBV. (See Hostetler et al., Biochem Pharmacol 53:1815-22 (1997); Painter et al., Antimicrob. Agents Chemother. 51:3505-9 (2007), and Lanier et al., AAC 2010).

### SUMMARY

A first aspect of the invention provides a pharmaceutical composition comprising a compound having a formula:

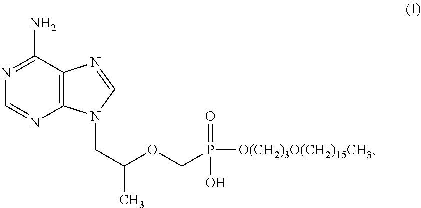

or a pharmaceutically acceptable salt, a stereoisomer, a diastereomer, an enantiomer or a racemate thereof in a dosage unit form comprising an amount from about 5 mg to about 100 mg of said compound, or pharmaceutically acceptable salt, stereoisomer, diastereomer, enantiomer or racemate thereof. For example, the unit dosage form can contain from about 5 mg to about 75 mg, from about 5 mg to about 50 mg, from about 5 mg to about 25 mg, or from about 5 mg to about 10 mg of said compound or pharmaceutically acceptable salt, stereoisomer, diastereomer, enantiomer or racemate thereof. For example, the unit dosage form can contain from about 10 mg to about 75 mg, from about 10 mg to about 50 mg, or from about 10 mg to about 25 mg of said compound or pharmaceutically acceptable salt, stereoisomer, diastereomer, enantiomer or racemate thereof. For example, the unit dosage form contains about 5 mg, about 10 mg, about 15 mg, about 20 mg, about 25 mg, about 50 mg, about 75 mg, or about 100 mg of said compound or pharmaceutically acceptable salt, stereoisomer, diastereomer, enantiomer or racemate thereof.

In some embodiments, the compound having a formula (I) is Compound I:

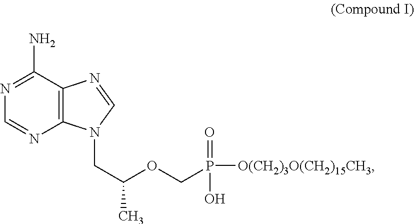

or a pharmaceutically acceptable salt thereof.

In some embodiments, the pharmaceutically acceptable salt of the compound of formula (I) is:

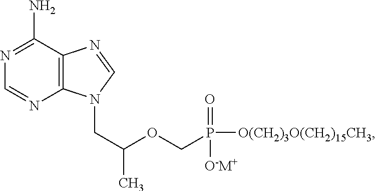

wherein M⁺ is Na⁺, Li⁺, K⁻, Ca²⁺, Mg²⁺, or NR_{c}R_{d}RₑR_{f}⁺ and R_{c}, R_{d}, Rₑ, R_{f} are each independently hydrogen or C₁₋₅ alkyl, or stereoisomer, a diastereomer, an enantiomer or racemate thereof. In some embodiments, M⁺ is Li⁺, K⁺, Ca²⁺, Mg²⁺or NR^{d}R^{e}R^{f}R^{g+} and R^{d}, R^{e}, and R^{f} are each independently hydrogen or C₁₋₅ alkyl. In some embodiments, M⁺ is Na⁺, Li⁺, K⁺, Ca²⁺, Mg²⁺, or NH₄⁺. In some embodiments, M⁺ is Li⁺, K⁺, Ca²⁺, Mg²⁺or NH₄⁺. In some embodiments, M⁺ is Na⁺, Li⁺, K⁺, or NH₄⁺. In some embodiments, M⁺ is Li⁺ or NH₄⁺. In some embodiments, M⁺ is K⁺. When M⁺ is Ca²⁺ or Mg²⁺, two equivalents of the anions are present so as to form neutral molecules.

In some embodiments, the pharmaceutically acceptable salt of the compound of formula (I) (e.g., of Compound I) is:

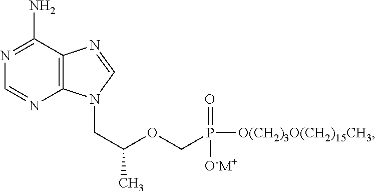

wherein M⁺ is as above defined.

In another aspect, the present disclosure provides a method for treating a viral infection in a subject, the method comprising administering to the subject a compound having a formula:

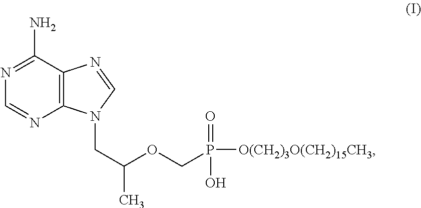

or a pharmaceutically acceptable salt, a stereoisomer, a diastereomer, an enantiomer or a racemate thereof, wherein said compound, or pharmaceutically acceptable salt, stereoisomer, diastereomer, enantiomer or racemate thereof, is administered to a subject at a dosage of about 5 mg to about 100 mg. For example, the method comprises administering to the subject from about 5 mg to about 75 mg, from about 5 mg to about 50 mg, from about 5 mg to about 25 mg, or from about 5 mg to about 10 mg of said compound or pharmaceutically acceptable salt, stereoisomer, diastereomer, enantiomer or racemate thereof (e.g., Compound I or a pharmaceutically acceptable salt thereof). For example, the method comprises administering to the subject from about 10 mg to about 75 mg, from about 10 mg to about 50 mg, or from about 10 mg to about 25 mg of said compound or pharmaceutically acceptable salt, stereoisomer, diastereomer, enantiomer or racemate thereof (e.g., Compound I or a pharmaceutically acceptable salt thereof). For example, the method comprises administering to the subject about 5 mg, about 10 mg, about 15 mg, about 20 mg, about 25 mg, about 50 mg, about 75 mg, or about 100 mg of said compound or pharmaceutically acceptable salt, stereoisomer, diastereomer, enantiomer or racemate thereof (e.g., Compound I or a pharmaceutically acceptable salt thereof).

In another aspect, the present disclosure provides a compound having a formula:

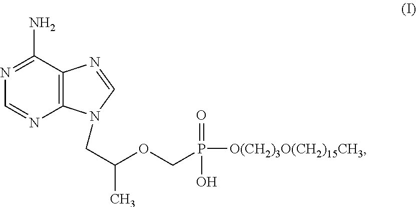

or a pharmaceutically acceptable salt, a stereoisomer, a diastereomer, an enantiomer or a racemate thereof, for use in treating a viral infection in a subject, wherein said compound, or pharmaceutically acceptable salt, stereoisomer, diastereomer, enantiomer or racemate thereof, is administered to the subject in an amount of about 5 mg, about 10 mg, about 15 mg, about 20 mg, about 25 mg, about 50 mg, about 75 mg, or about 100 mg. In some embodiments, the compound is Compound I, or a pharmaceutically acceptable salt thereof.

In another aspect, the present disclosure provides a compound having a formula:

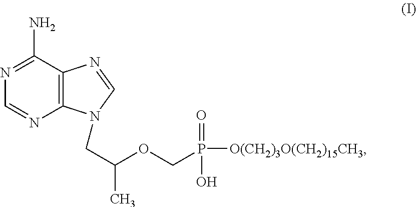

or a pharmaceutically acceptable salt, a stereoisomer, a diastereomer, an enantiomer or a racemate thereof, for use in the manufacture of a medicament for treating a viral infection in a subject, wherein the medicament comprises said compound, or pharmaceutically acceptable salt, stereoisomer, diastereomer, enantiomer or racemate thereof, in an amount of about 5 mg, about 10 mg, about 15 mg, about 20 mg, about 25 mg, about 50 mg, about 75 mg, or about 100 mg. In some embodiments, the compound is Compound I, or a pharmaceutically acceptable salt thereof.

In another aspect, the present disclosure provides for the use of a compound having a formula:

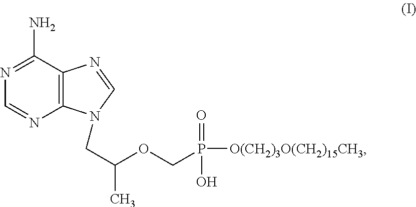

or a pharmaceutically acceptable salt, a stereoisomer, a diastereomer, an enantiomer or a racemate thereof, in treating a viral infection in a subject, wherein said compound or pharmaceutically acceptable salt, stereoisomer, diastereomer, enantiomer or racemate thereof, is administered to the subject in an amount of about 5 mg, about 10 mg, about 15 mg, about 20 mg, about 25 mg, about 50 mg, about 75 mg, or about 100 mg. In some embodiments, the compound is Compound I, or a pharmaceutically acceptable salt thereof.

In another aspect, the present disclosure provides for the use of a compound having a formula:

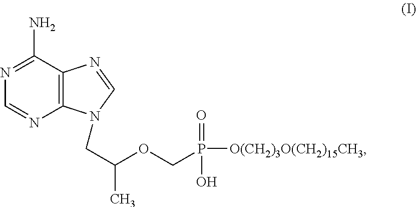

or a pharmaceutically acceptable salt, a stereoisomer, a diastereomer, an enantiomer or a racemate thereof, in the manufacture of a medicament for treating a viral infection in a subject, wherein the medicament comprises said compound, or pharmaceutically acceptable salt, stereoisomer, diastereomer, enantiomer or racemate thereof, in an amount of about 5 mg, about 10 mg, about 15 mg, about 20 mg, about 25 mg, about 50 mg, about 75 mg, or about 100 mg. In some embodiments, the compound is Compound I, or a pharmaceutically acceptable salt thereof.

### BRIEF DESCRIPTION OF THE DRAWINGS

FIG. 1A is graph illustrating the change from baseline in systolic blood pressure over time, during and after oral administration of 5 mg of Compound I daily. Error bars represent the 25^{th} and 75th percentiles. The 6-digit numbers denoting each curve identify the corresponding patient.

FIG. 1B is graph illustrating the change from baseline in diastolic blood pressure over time, during and after oral administration of 5 mg of Compound I daily. Compound I was administered orally to 8 different subjects for up to 14 days. Error bars represent the 25^{th} and 75^{th} percentiles. The 6-digit numbers denoting each curve identify the corresponding patient.

FIG. 2A is a graph illustrating the change from baseline in systolic blood pressure over time, during and after oral administration of 10 mg of Compound I daily. Error bars represent the 25^{th} and 75^{th} percentiles. The 6-digit numbers denoting each curve identify the corresponding patient.

FIG. 2B is a graph illustrating the change from baseline in diastolic blood pressure over time, during and after oral administration of 10 mg of Compound I daily. Error bars represent the 25^{th} and 75^{th} percentiles. The 6-digit numbers denoting each curve identify the corresponding patient.

FIG. 3A is a graph illustrating the change from baseline in systolic blood pressure over time, during and after oral administration of a placebo. Error bars represent the 25^{th} and 75^{th} percentiles. The 6-digit numbers denoting each curve identify the corresponding patient.

FIG. 3B is a graph illustrating the change from baseline in diastolic blood pressure over time, during and after oral administration of a placebo. Error bars represent the 25^{th} and 75^{th} percentiles. The 6-digit numbers denoting each curve identify the corresponding patient.

FIG. 4A is a graph illustrating the change from baseline in heart rate over time, during and after oral administration of 5 mg of Compound I daily. Error bars represent the 25^{th} and 75^{th} percentiles. The 6-digit numbers denoting each curve identify the corresponding patient.

FIG. 4B is a graph illustrating the change from baseline in RR interval over time, during and after oral administration of 5 mg of Compound I daily. Error bars represent the 25^{th} and 75^{th} percentiles. The 6-digit numbers denoting each curve identify the corresponding patient.

FIG. 5A is a graph illustrating the change from baseline in heart rate over time, during and after oral administration of 10 mg of Compound I daily. Error bars represent the 25^{th} and 75^{th} percentiles. The 6-digit numbers denoting each curve identify the corresponding patient.

FIG. 5B is a graph illustrating the change from baseline in RR interval over time, during and after oral administration of 10 mg of Compound I daily. Error bars represent the 25^{th} and 75^{th} percentiles. The 6-digit numbers denoting each curve identify the corresponding patient.

FIG. 6A is a graph illustrating the change from baseline in heart rate over time, during and after oral administration of a placebo. Error bars represent the 25^{th} and 75^{th} percentiles. The 6-digit numbers denoting each curve identify the corresponding patient.

FIG. 6B is a graph illustrating the change from baseline in RR interval over time, during and after oral administration of a placebo. Error bars represent the 25^{th} and 75^{th} percentiles. The 6-digit numbers denoting each curve identify the corresponding patient.

FIG. 7A is a graph illustrating the change from baseline in PR interval over time, during and after oral administration of 5 mg of Compound I daily. Error bars represent the 25^{th} and 75^{th} percentiles. The 6-digit numbers denoting each curve identify the corresponding patient.

FIG. 7B is a graph illustrating the change from baseline in QRS duration over time, during and after oral administration of 5 mg of Compound I daily. Error bars represent the 25th and 75th percentiles. The 6-digit numbers denoting each curve identify the corresponding patient.

FIG. 7C is a graph illustrating the change from baseline in QTcF over time, during and after oral administration of 5 mg of Compound I daily. Error bars represent the 25^{th} and 75^{th} percentiles. The 6-digit numbers denoting each curve identify the corresponding patient.

FIG. 8A is a graph illustrating the change from baseline in PR interval over time, during and after oral administration of 10 mg of Compound I daily. Error bars represent the 25^{th} and 75^{th} percentiles. The 6-digit numbers denoting each curve identify the corresponding patient.

FIG. 8B is a graph illustrating the change from baseline in QRS duration over time, during and after oral administration of 10 mg of Compound I daily. Error bars represent the 25th and 75th percentiles. The 6-digit numbers denoting each curve identify the corresponding patient.

FIG. 8C is a graph illustrating the change from baseline in QTcF over time, during and after oral administration of 10 mg of Compound I daily. Error bars represent the 25^{th} and 75^{th} percentiles. The 6-digit numbers denoting each curve identify the corresponding patient.

FIG. 9A is a graph illustrating the change from baseline in PR interval over time, during and after oral administration of a placebo. Error bars represent the 25^{th} and 75^{th} percentiles. The 6-digit numbers denoting each curve identify the corresponding patient.

FIG. 9B is a graph illustrating the change from baseline in QRS duration over time, during and after oral administration of a placebo. Error bars represent the 25^{th} and 75^{th} percentiles. The 6-digit numbers denoting each curve identify the corresponding patient.

FIG. 9C is a graph illustrating the change from baseline in QTcF over time, during and after oral administration of a placebo. Error bars represent the 25^{th} and 75^{th} percentiles. The 6-digit numbers denoting each curve identify the corresponding patient.

### DETAILED DESCRIPTION

The present disclosure provides compounds and methods for the treatment of a viral infection. In some embodiments, the virus is in the family Hepadnaviridae. In some embodiments, the virus is hepatitis B (HBV).

In one aspect, the present disclosure provides a method of treating viral infection, e.g., HBV infection, the method comprising administering to a subject in need thereof an effective amount of a compound having a formula (I), or a pharmaceutically acceptable salt, a stereoisomer, a diastereomer, an enantiomer or a racemate thereof (e.g., Compound I or a pharmaceutically acceptable salt thereof).

In one aspect the present disclosure provides a compound having a formula (I), or a pharmaceutically acceptable salt, a stereoisomer, a diastereomer, an enantiomer or a racemate thereof (e.g., Compound I or a pharmaceutically acceptable salt thereof), for use in treating a viral infection in a subject, wherein said compound, or pharmaceutically acceptable salt, stereoisomer, diastereomer, enantiomer or racemate thereof, is administered to the subject in an amount of about 5 mg, about 10 mg, about 15 mg, about 20 mg, about 25 mg, about 50 mg, about 75 mg, or about 100 mg. In one embodiment, the compound having a formula (I), or a pharmaceutically acceptable salt, a stereoisomer, a diastereomer, an enantiomer or a racemate thereof (e.g., Compound I or a pharmaceutically acceptable salt thereof), is administered to the subject in an amount from about 5 mg to about 75 mg, from about 5 mg to about 50 mg, from about 5 mg to about 25 mg, or from about 5 mg to about 10 mg. In one embodiment, the compound having a formula (I), or a pharmaceutically acceptable salt, a stereoisomer, a diastereomer, an enantiomer or a racemate thereof (e.g., Compound I or a pharmaceutically acceptable salt thereof), is administered to the subject in an amount from about 10 mg to about 75 mg, from about 10 mg to about 50 mg, or from about 10 mg to about 25.

In one aspect the present disclosure provides a compound having a formula (I), or a pharmaceutically acceptable salt, a stereoisomer, a diastereomer, an enantiomer or a racemate thereof (e.g., Compound I or a pharmaceutically acceptable salt thereof), for use in the manufacture of a medicament for treating a viral infection in a subject, wherein the medicament comprises said compound, or pharmaceutically acceptable salt, stereoisomer, diastereomer, enantiomer or racemate thereof, in an amount of about 5 mg, about 10 mg, about 15 mg, about 20 mg, about 25 mg, about 50 mg, about 75 mg, or about 100 mg. In one embodiment, the medicament comprises the compound having a formula (I), or a pharmaceutically acceptable salt, a stereoisomer, a diastereomer, an enantiomer or a racemate thereof (e.g., Compound I or a pharmaceutically acceptable salt thereof), in an amount from about 5 mg to about 75 mg, from about 5 mg to about 50 mg, from about 5 mg to about 25 mg, or from about 5 mg to about 10 mg. In one embodiment, the medicament comprises the compound having a formula (I), or a pharmaceutically acceptable salt, a stereoisomer, a diastereomer, an enantiomer or a racemate thereof (e.g., Compound I or a pharmaceutically acceptable salt thereof), in an amount from about 10 mg to about 75 mg, from about 10 mg to about 50 mg, or from about 10 mg to about 25.

In another aspect, the present disclosure provides a compound having a formula (I), or a pharmaceutically acceptable salt, a stereoisomer, a diastereomer, an enantiomer or a racemate thereof (e.g., Compound I or a pharmaceutically acceptable salt thereof) for use in the manufacture of a medicament for treating a viral infection in a subject, wherein the medicament comprises said compound, or pharmaceutically acceptable salt, stereoisomer, diastereomer, enantiomer or racemate thereof, in an amount of about 5 mg, about 10 mg, about 15 mg, about 20 mg, about 25 mg, about 50 mg, about 75 mg, or about 100 mg. In one embodiment, the medicament comprises the compound having a formula (I), or a pharmaceutically acceptable salt, a stereoisomer, a diastereomer, an enantiomer or a racemate thereof (e.g., Compound I or a pharmaceutically acceptable salt thereof), in an amount from about 5 mg to about 75 mg, from about 5 mg to about 50 mg, from about 5 mg to about 25 mg, or from about 5 mg to about 10 mg. In one embodiment, the medicament comprises the compound having a formula (I), or a pharmaceutically acceptable salt, a stereoisomer, a diastereomer, an enantiomer or a racemate thereof (e.g., Compound I or a pharmaceutically acceptable salt thereof), in an amount from about 10 mg to about 75 mg, from about 10 mg to about 50 mg, or from about 10 mg to about 25.

In another aspect, the present disclosure provides the use of a compound having a formula (I), or a pharmaceutically acceptable salt, a stereoisomer, a diastereomer, an enantiomer or a racemate thereof (e.g., Compound I or a pharmaceutically acceptable salt thereof), in treating a viral infection in a subject, wherein said compound, or pharmaceutically acceptable salt, stereoisomer, diastereomer, enantiomer or racemate thereof, is administered to the subject in an amount of about 5 mg, about 10 mg, about 15 mg, about 20 mg, about 25 mg, about 50 mg, about 75 mg, or about 100 mg. In one embodiment, the compound having a formula (I), or a pharmaceutically acceptable salt, a stereoisomer, a diastereomer, an enantiomer or a racemate thereof (e.g., Compound I or a pharmaceutically acceptable salt thereof), is administered to the subject in an amount from about 5 mg to about 75 mg, from about 5 mg to about 50 mg, from about 5 mg to about 25 mg, or from about 5 mg to about 10 mg. In one embodiment, the compound having a formula (I), or a pharmaceutically acceptable salt, a stereoisomer, a diastereomer, an enantiomer or a racemate thereof (e.g., Compound I or a pharmaceutically acceptable salt thereof), is administered to the subject in an amount from about 10 mg to about 75 mg, from about 10 mg to about 50 mg, or from about 10 mg to about 25.

In another aspect, the present disclosure provides the use of a compound having a formula (I), or a pharmaceutically acceptable salt, a stereoisomer, a diastereomer, an enantiomer or a racemate thereof (e.g., Compound I or a pharmaceutically acceptable salt thereof), in the manufacture of a medicament for treating a viral infection in a subject, wherein the medicament comprises said compound, or pharmaceutically acceptable salt, stereoisomer, diastereomer, enantiomer or racemate thereof, in an amount of about 5 mg, about 10 mg, about 15 mg, about 20 mg, about 25 mg, about 50 mg, about 75 mg, or about 100 mg. In one embodiment, the medicament comprises the compound having a formula (I), or a pharmaceutically acceptable salt, a stereoisomer, a diastereomer, an enantiomer or a racemate thereof (e.g., Compound I or a pharmaceutically acceptable salt thereof), in an amount from about 5 mg to about 75 mg, from about 5 mg to about 50 mg, from about 5 mg to about 25 mg, or from about 5 mg to about 10 mg. In one embodiment, the medicament comprises the compound having a formula (I), or a pharmaceutically acceptable salt, a stereoisomer, a diastereomer, an enantiomer or a racemate thereof (e.g., Compound I or a pharmaceutically acceptable salt thereof), in an amount from about 10 mg to about 75 mg, from about 10 mg to about 50 mg, or from about 10 mg to about 25.

The present disclosure also provides pharmaceutical compositions comprising a compound having a formula (I), or a pharmaceutically acceptable salt, a stereoisomer, a diastereomer, an enantiomer or a racemate thereof (e.g., Compound I or a pharmaceutically acceptable salt thereof), and a pharmaceutically acceptable carrier.

As set forth in the present specification, daily doses of 5 mg and 10 mg for 9-14 days were both safe and well-tolerated. The presence of Compound I and/or tenofovir in patients' plasma after 24 hours following administration at 5 mg suggests that this dosage range is feasible for daily or less frequent administration.

In some embodiments, the methods of the present invention provide higher in vivo plasma concentrations of active antiviral (i.e., tenofovir diphosphate) using lower dosages of the compound having a formula (I), or a pharmaceutically acceptable salt, a stereoisomer, a diastereomer, an enantiomer or a racemate thereof (e.g. Compound I, or a pharmaceutically acceptable salt thereof) compared with administration of tenofovir.

### Definitions

For the purposes of promoting an understanding of the embodiments described herein specific language is used to describe the same. The terminology used herein is for the purpose of describing particular embodiments only, and is not intended to limit the scope of the present invention. As used throughout this disclosure, the singular forms “a,” “an,” and “the” include plural reference unless the context clearly dictates otherwise. Thus, for example, a reference to “a composition” includes a plurality of such compositions, as well as a single composition, and a reference to “a therapeutic agent” or “an active compound” is a reference to the therapeutic and/or pharmaceutical agent (e.g., a compound having formula (I), or a pharmaceutically acceptable salt, a stereoisomer, a diastereomer, an enantiomer or a racemate thereof, e.g., Compound I or a pharmaceutically acceptable salt thereof) and equivalents thereof known to those skilled in the art. All percentages and ratios used herein, unless otherwise indicated, are by weight.

The term “about” is used herein to mean approximately, in the region of, roughly or around. When the term “about” is used in conjunction with a numerical range, it modifies that range by extending the boundaries above and below the numerical values set forth. In general, the term “about” is used herein to modify a numerical value above and below the stated value by a variance of 5%.

As used in the present disclosure, whether in a transitional phrase or in the body of a claim, the terms “comprise(s)” and “comprising” are to be interpreted as having an open-ended meaning. That is, the terms are to be interpreted synonymously with the phrases “having at least” or “including at least.” When used in the context of a process the term “comprising” means that the process includes at least the recited steps, but may include additional steps. When used in the context of a molecule, compound, or composition, the term “comprising” means that the compound or composition includes at least the recited features or components, but may also include additional features or components.

A “pharmaceutical composition” is a formulation containing the compound of the present invention (i.e., a compound having formula (I), or a pharmaceutically acceptable salt, a stereoisomer, a diastereomer, an enantiomer or a racemate thereof, e.g., Compound I or a pharmaceutically acceptable salt thereof) in a form suitable for administration to a subject. The term “pharmaceutical composition” includes preparations suitable for administration to mammals, e.g., humans.

As used herein, “treating,” “treatment” or “treat” describes the management and care of a patient for the purpose of combating a disease, condition, or disorder and includes the administration of active compounds of the present invention (i.e., a compound having a formula (I), or a pharmaceutically acceptable salt, a stereoisomer, a diastereomer, an enantiomer or a racemate thereof, e.g. Compound I, or a pharmaceutically acceptable salt thereof), to lessen or alleviate the symptoms or complications of a disease, condition or disorder, and in some cases, to eliminate the disease, condition or disorder. The term “treat” can also include treatment of a cell in vitro or an animal model.

As used herein, “therapeutically effective amount” means that amount necessary to make a clinically observed improvement in the patient. In some embodiments, the compounds of the disclosure are formulated such that they comprise an amount that would not cause one or more unwanted side effects. For instance, the term “therapeutically effective amount”, can refer to an amount of any pharmaceutical agent or agents (e.g., a compound having a formula (I), or a pharmaceutically acceptable salt, a stereoisomer, a diastereomer, an enantiomer or a racemate thereof, e.g., Compound I or a pharmaceutically acceptable salt thereof) to treat, or ameliorate an identified disease or condition (e.g., HBV infection), or to exhibit a detectable therapeutic or inhibitory effect. The effect can be detected by any assay method known in the art. The precise effective amount for a subject will depend upon the subject's body weight, size, and health; the nature and extent of the condition; and the dosing schedule of therapeutics selected for administration.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient or carrier” means an excipient or carrier that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

For instance, a “pharmaceutically acceptable carrier” may include any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Except insofar as any conventional carrier medium is incompatible with the active compound (i.e., a compound having a formula (I), or a pharmaceutically acceptable salt, a stereoisomer, a diastereomer, an enantiomer or a racemate thereof, e.g., Compound I or a pharmaceutically acceptable salt thereof) such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; buffering agents such as magnesium hydroxide and aluminum hydroxide; alginic acid; pyrogen free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

As used herein, a “subject” is interchangeable with a “subject in need thereof”, both of which refer to a subject (e.g., a patient) having a disorder in which viral infection plays a part. A “subject” includes a mammal. The mammal can be e.g., a human or appropriate non-human mammal, such as a non-human primate, mouse, rat, dog, cat, cow, horse, goat, camel, sheep, chicken or a pig. In some embodiments, the mammal is a human.

As used herein, the expression “daily” is meant to comprise the term “once per day” as well as “every day”. Similarly, as used herein, the expression “weekly” is meant to comprise the term “once per week” as well as “every week”.

### Compound I

The structure of HDP-tenofovir, also known as HDP-TFV or Compound I is given below:

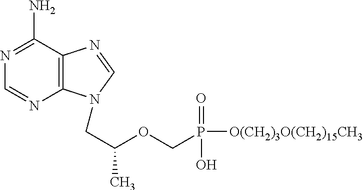

In some embodiments, the pharmaceutical composition comprises a salt of Compound I, for instance:

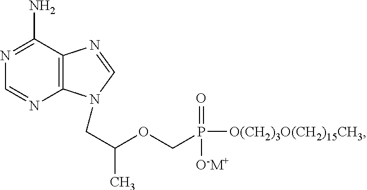

wherein M⁺ is Na⁺, Li⁺, K⁺, Ca²⁺, Mg²⁺, or NR_{c}R_{d}RₑR_{f}⁺ and R_{c}, R_{d}, Rₑ, R_{f} are each independently hydrogen or C₁₋₅ alkyl, or stereoisomer, a diastereomer, an enantiomer or racemate thereof. In some embodiments, M⁺ is Li⁺, K⁺, Ca²⁺, Mg²⁺or NR^{d}R^{e}R^{f}R^{g+} and R^{d}, R^{e}, and R^{f} are each independently hydrogen or C₁₋₅ alkyl. In some embodiments, M⁺ is Na⁺, Li⁺, K⁺, Ca²⁺, Mg²⁻, or NH₄⁺. In some embodiments, M⁺ is Li⁺, K⁺, Ca²⁺, Mg²⁺or NH₄⁺. In some embodiments, M⁺ is Na⁺, Li⁺, K⁺, or NH₄⁺. In some embodiments, M⁺ is Li⁻ or NH₄⁺. In some embodiments, M⁺ is K⁺. When M⁺ is Ca²⁺ or Mg²⁺, two equivalents of the anions are present so as to form neutral molecules.

In some embodiments, the pharmaceutically acceptable salt of the compound of formula (I) (e.g., of Compound I) is:

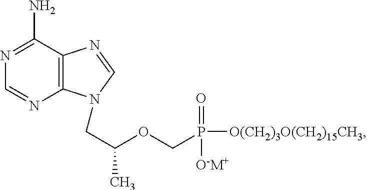

wherein M⁺ is as above defined.

Compound I is a lipid conjugate antiviral agent that mimics natural phospholipids and is efficiently absorbed in the intestine, is not cleaved in peripheral circulation, and is uptaken into target cells where it can be converted to tenofovir and ultimately tenofovir diphosphate (TFV-PP):

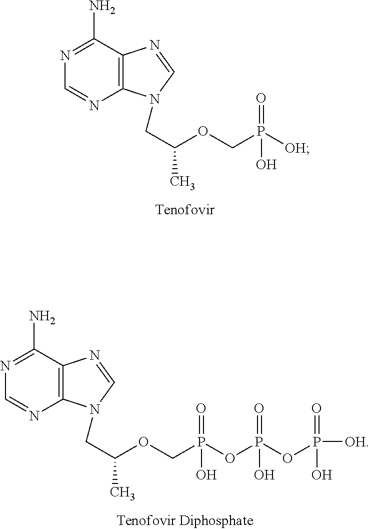 [SMILES: C[C@H](Cn1cnc2c(N)ncnc21)OCP(=O)(O)O ; C[C@H](Cn1cnc2c(N)ncnc21)OCP(=O)(O)OP(=O)(O)OP(=O)(O)O]

### Methods of the Disclosure

The present disclosure relates to methods of treating HBV infection administering to a subject in need thereof (e.g., a subject who has HBV) a compound having a formula (I), or a pharmaceutically acceptable salt, a stereoisomer, a diastereomer, an enantiomer or a racemate thereof (e.g. Compound I or a pharmaceutically acceptable salt thereof) at a dosage of between about 5 mg and about 100 mg (e.g., about 5 mg, about 10 mg, about 15 mg, about 20 mg, about 25 mg, about 50 mg, about 75 mg, or about 100 mg). In some embodiments, the dosages are safe and effective and give rise to plasma levels of HDP-TFV that are sufficient to treat HBV.

In some embodiments, a compound of the disclosure (i.e., a compound having a formula (I), or a pharmaceutically acceptable salt, a stereoisomer, a diastereomer, an enantiomer or a racemate thereof, e.g. Compound I, or a pharmaceutically acceptable salt thereof), is administered at a dosage of from about 5 mg to about 100 mg. For example, a compound having a formula (I), or a pharmaceutically acceptable salt, a stereoisomer, a diastereomer, an enantiomer or a racemate thereof, (e.g., Compound I, or a pharmaceutically acceptable salt thereof), is administered at a dosage of from about 5 mg to about 75 mg, from about 5 mg to about 50 mg, from about 5 mg to about 25 mg, or from about 5 mg to about 10 mg. For example, a compound having a formula (I), or a pharmaceutically acceptable salt, a stereoisomer, a diastereomer, an enantiomer or a racemate thereof, (e.g., Compound I, or a pharmaceutically acceptable salt thereof), is administered at a dosage of from about 10 mg to about 75 mg, from about 10 mg to about 50 mg, or from about 10 mg to about 25 mg. For example, said compound or pharmaceutically acceptable salt, stereoisomer, diastereomer, enantiomer or racemate thereof is administered to said subject in an amount of 5, 6, 7, 8, 9, 10, 11, 12, 13, 14, 15, 20, 25, 30, 35, 40, 45, 50, 55, 60, 65, 70, 75, 80, 85, 90, 95 or 100 mg. The compounds of the disclosure (i.e., a compound having a formula (I), or a pharmaceutically acceptable salt, a stereoisomer, a diastereomer, an enantiomer or a racemate thereof, e.g. Compound I, or a pharmaceutically acceptable salt thereof), can be administered, for example, as a single dose, daily, or weekly. In some embodiments, the subject is a human being. In some embodiments, the compound is administered orally.

In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered to a subject at a dose of about 5 mg. In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered to a subject at a dose of about 10 mg. In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered to a subject at a dose of about 15 mg. In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered to a subject at a dose of about 20 mg. In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered to a subject at a dose of about 25 mg. In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered to a subject at a dose of about 50 mg. In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered to a subject at a dose of about 75 mg. In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered to a subject at a dose of about 100 mg. In some embodiments, the compound having a formula (I), or a pharmaceutically acceptable salt, a stereoisomer, a diastereomer, an enantiomer or a racemate thereof (e.g. Compound I, or a pharmaceutically acceptable salt thereof), is administered orally.

In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered for more than a month. In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered for more than six months. In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered for more than a year. In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered for more than five years. In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered for more than ten years. In some embodiments, the compound having a formula (I), or a pharmaceutically acceptable salt, a stereoisomer, a diastereomer, an enantiomer or a racemate thereof (e.g. Compound I, or a pharmaceutically acceptable salt thereof), is administered for more than 25 years. In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered for the duration of the subject's life.

In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered for less than a year. In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered for less than five years. In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered for less than ten years. In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered for less than 25 years.

In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered for up to a year. In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered for up to five years. In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered for up to ten years. In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered for up to 25 years.

In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered for a duration of time between one day and one week. In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered for a duration of time between one day and one month. In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered for a duration of time between one day and one year. In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered for a duration of time between one day and five years. In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered for a duration of time between one day and ten years. In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered for a duration of time between one day and 25 years. In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered for a duration of time between one day and the duration of the subject's life.

In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered for a duration of time between one week and one month. In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered for a duration of time between one week and one year. In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered for a duration of time between one week and five years. In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered for a duration of time between one week and ten years. In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered for a duration of time between one week and 25 years. In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered for a duration of time between one week and the duration of the subject's life.

In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered for a duration of time between one month and one year. In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered for a duration of time between one month and five years. In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered for a duration of time between one month and ten years. In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered for a duration of time between one month and 25 years. In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered for a duration of time between one month and the duration of the subject's life.

In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered for a duration of time between one year and five years. In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered for a duration of time between one year and ten years. In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered for a duration of time between one year and 25 years. In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered for a duration of time between one year and the duration of the subject's life.

In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered for a duration of time between five years and ten years. In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered for a duration of time between five years and 25 years. In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered for a duration of time between five years and the duration of the subject's life.

In some embodiments, the duration of treatment is independent of the frequency of treatment. That is, a compound having a formula (I), or a pharmaceutically acceptable salt, a stereoisomer, a diastereomer, an enantiomer or a racemate thereof (e.g., Compound I, or a pharmaceutically acceptable salt thereof) can be administered for example, weekly for six months, or daily for the duration of the subject's life, or in any combination of the treatment durations, administration frequencies and dosages mentioned herein. In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered orally.

In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered daily. In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered daily for 1, 2, 3, 4, 5, 6, 7, 8, 9, 10, 11, 12, 13, 14, 15, 16, 17, 18, 19, 20, 21, 22, 23, 24, 25, 26, 27, or 28 days. In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered weekly. In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered weekly for 1, 2, 3, 4, 5, 6, 7, 8, 9, 10, 11, 12, 13, 14, 15, 16, 17, 18, 19, 20, 21, 22, 23, 24, 25, 26, 28, 29, 30, 31, 32, 33, 34, 35, 36, 37, 38, 39, 40, 41, 42, 43, 44, 45, 46, 47, or 48 weeks.

In some embodiments, the dosage unit form is in the form of an orally administered pill, capsule or tablet. In some embodiments, the dosage unit form is in the form of an orally administered suspension, solution, emulsion, mouthwash or elixir.

In some embodiments, active compounds of the present disclosure (i.e., a compound having a formula (I), or a pharmaceutically acceptable salt, a stereoisomer, a diastereomer, an enantiomer or a racemate thereof, e.g., Compound I, or a pharmaceutically acceptable salt thereof) is administered to a subject as a single dose. In some embodiments, a compound having a formula (I), or a pharmaceutically acceptable salt, a stereoisomer, a diastereomer, an enantiomer or a racemate thereof (e.g., Compound I, or a pharmaceutically acceptable salt thereof), is administered to a subject in multiple doses. Multiple doses of a compound having a formula (I), or a pharmaceutically acceptable salt, a stereoisomer, a diastereomer, an enantiomer or a racemate thereof (e.g., Compound I, or a pharmaceutically acceptable salt thereof), is administered regularly, for example, once every 8 hours, once every 12 hours or once per day. For example, a compound having a formula (I), or a pharmaceutically acceptable salt, a stereoisomer, a diastereomer, an enantiomer or a racemate thereof (e.g., Compound I, or a pharmaceutically acceptable salt thereof), is administered once or multiple times per day for any duration of time. For example, the compound having a formula (I), or a pharmaceutically acceptable salt, a stereoisomer, a diastereomer, an enantiomer or a racemate thereof (e.g., Compound I, or a pharmaceutically acceptable salt thereof), is administered once, two, three or four times or more per day. For example, doses is administered once, two, three, four, five or six times, or more times per week. Moreover, each individual dose is administered with the same or a different dosage.

In some embodiments, a compound having a formula (I), or a pharmaceutically acceptable salt, a stereoisomer, a diastereomer, an enantiomer or a racemate thereof (e.g., Compound I, or a pharmaceutically acceptable salt thereof), is administered, for example, once per day for any duration of time. For example, a compound having a formula (I), or a pharmaceutically acceptable salt, a stereoisomer, a diastereomer, an enantiomer or a racemate thereof (e.g., Compound I, or a pharmaceutically acceptable salt thereof), is administered once per day for 1, 2, 3, 4, 5, 6, 7, 8, 9, 10, 11, 12, 13, 14, 15, 16, 17, 18, 19, 20, 21, 22, 23, 24, 25, 26, 27, or 28 days or more. In some embodiments, the compound having a formula (I), or a pharmaceutically acceptable salt, a stereoisomer, a diastereomer, an enantiomer or a racemate thereof (e.g., Compound I, or a pharmaceutically acceptable salt thereof), is administered two, three, or four or more times per day for 1, 2, 3, 4, 5, 6, 7, 8, 9, 10, 11, 12, 13, 14, 15, 16, 17, 18, 19, 20, 21, 22, 23, 24, 25, 26, 27, or 28 days. For example, the compound having a formula (I), or a pharmaceutically acceptable salt, a stereoisomer, a diastereomer, an enantiomer or a racemate thereof (e.g., Compound I, or a pharmaceutically acceptable salt thereof), is administered two times per day for 1, 2, 3, 4, 5, 6, 7, 8, 9, 10, 11, 12, 13, 14, 15, 16, 17, 18, 19, 20, 21, 22, 23, 24, 25, 26, 27, or 28 days. For example, the compound having a formula (I), or a pharmaceutically acceptable salt, a stereoisomer, a diastereomer, an enantiomer or a racemate thereof (e.g., Compound I, or a pharmaceutically acceptable salt thereof), is administered three times per day for 1, 2, 3, 4, 5, 6, 7, 8, 9, 10, 11, 12, 13, 14, 15, 16, 17, 18, 19, 20, 21, 22, 23, 24, 25, 26, 27, or 28 days. For example, the compound having a formula (I), or a pharmaceutically acceptable salt, a stereoisomer, a diastereomer, an enantiomer or a racemate thereof (e.g., Compound I, or a pharmaceutically acceptable salt thereof), is administered four times per day for 1, 2, 3, 4, 5, 6, 7, 8, 9, 10, 11, 12, 13, 14, 15, 16, 17, 18, 19, 20, 21, 22, 23, 24, 25, 26, 27, or 28 days. The pharmaceutical compositions of the disclosure can be administered indefinitely to suppress a viral infection (e.g., HBV). That is, the pharmaceutical compositions can be taken (e.g., daily), for months, years, or for the rest of the subject's life.

For example, 5 mg of a compound having a formula (I), or a pharmaceutically acceptable salt, a stereoisomer, a diastereomer, an enantiomer or a racemate thereof (e.g. Compound I, or a pharmaceutically acceptable salt thereof) is administered daily (e.g. once per day, or two, three, or four times or more per day). For example, 10 mg of a compound having a formula (I), or a pharmaceutically acceptable salt, a stereoisomer, a diastereomer, an enantiomer or a racemate thereof (e.g. Compound I, or a pharmaceutically acceptable salt thereof), is administered daily (e.g. once per day, or two, three, or four times or more per day). For example, 25 mg of a compound having a formula (I), or a pharmaceutically acceptable salt, a stereoisomer, a diastereomer, an enantiomer or a racemate thereof (e.g. Compound I, or a pharmaceutically acceptable salt thereof), is administered daily (e.g. once per day, or two, three, or four times or more per day). For example, 50 mg of a compound having a formula (I), or a pharmaceutically acceptable salt, a stereoisomer, a diastereomer, an enantiomer or a racemate thereof (e.g. Compound I, or a pharmaceutically acceptable salt thereof), is administered daily (e.g. once per day, or two, three, or four times or more per day). For example, 75 mg of a compound having a formula (I), or a pharmaceutically acceptable salt, a stereoisomer, a diastereomer, an enantiomer or a racemate thereof (e.g. Compound I, or a pharmaceutically acceptable salt thereof), is administered daily (e.g. once per day, or two, three, or four times or more per day). For example, 100 mg of a compound having a formula (I), or a pharmaceutically acceptable salt, a stereoisomer, a diastereomer, an enantiomer or a racemate thereof (e.g. Compound I, or a pharmaceutically acceptable salt thereof), is administered daily (e.g. once per day, or two, three, or four times or more per day).

In some embodiments, a compound having a formula (I), or a pharmaceutically acceptable salt, a stereoisomer, a diastereomer, an enantiomer or a racemate thereof (e.g. Compound I, or a pharmaceutically acceptable salt thereof) is administered, for example, once per week for any duration of time. In some embodiments, a compound having a formula (I), or a pharmaceutically acceptable salt, a stereoisomer, a diastereomer, an enantiomer or a racemate thereof (e.g. Compound I, or a pharmaceutically acceptable salt thereof) is administered once per week for 1, 2, 3, 4, 5, 6, 7, 8, 9, 10, 11, 12, 13, 14, 15, 16, 17, 18, 19, 20, 21, 22, 23, 24, 25, 26, 28, 29, 30, 31, 32, 33, 34, 35, 36, 37, 38, 39, 40, 41, 42, 43, 44, 45, 46, 47, or 48 weeks or more. In some embodiments, a compound having a formula (I), or a pharmaceutically acceptable salt, a stereoisomer, a diastereomer, an enantiomer or a racemate thereof (e.g., Compound I, or a pharmaceutically acceptable salt thereof) is administered two, three, four, five, or six times or more per week for 1, 2, 3, 4, 5, 6, 7, 8, 9, 10, 11, 12, 13, 14, 15, 16, 17, 18, 19, 20, 21, 22, 23, 24, 25, 26, 28, 29, 30, 31, 32, 33, 34, 35, 36, 37, 38, 39, 40, 41, 42, 43, 44, 45, 46, 47, or 48 weeks or more. For example, a compound having a formula (I), or a pharmaceutically acceptable salt, a stereoisomer, a diastereomer, an enantiomer or a racemate thereof (e.g., Compound I, or a pharmaceutically acceptable salt thereof) is administered two times per week for 1, 2, 3, 4, 5, 6, 7, 8, 9, 10, 11, 12, 13, 14, 15, 16, 17, 18, 19, 20, 21, 22, 23, 24, 25, 26, 28, 29, 30, 31, 32, 33, 34, 35, 36, 37, 38, 39, 40, 41, 42, 43, 44, 45, 46, 47, or 48 weeks or more. For example, a compound having a formula (I), or a pharmaceutically acceptable salt, a stereoisomer, a diastereomer, an enantiomer or a racemate thereof (e.g., Compound I, or a pharmaceutically acceptable salt thereof) is administered three times per week for 1, 2, 3, 4, 5, 6, 7, 8, 9, 10, 11, 12, 13, 14, 15, 16, 17, 18, 19, 20, 21, 22, 23, 24, 25, 26, 28, 29, 30, 31, 32, 33, 34, 35, 36, 37, 38, 39, 40, 41, 42, 43, 44, 45, 46, 47, or 48 weeks or more. For example, a compound having a formula (I), or a pharmaceutically acceptable salt, a stereoisomer, a diastereomer, an enantiomer or a racemate thereof (e.g., Compound I, or a pharmaceutically acceptable salt thereof) is administered four times per week for 1, 2, 3, 4, 5, 6, 7, 8, 9, 10, 11, 12, 13, 14, 15, 16, 17, 18, 19, 20, 21, 22, 23, 24, 25, 26, 28, 29, 30, 31, 32, 33, 34, 35, 36, 37, 38, 39, 40, 41, 42, 43, 44, 45, 46, 47, or 48 weeks or more. For example, a compound having a formula (I), or a pharmaceutically acceptable salt, a stereoisomer, a diastereomer, an enantiomer or a racemate thereof (e.g., Compound I, or a pharmaceutically acceptable salt thereof) is administered five times per week for 1, 2, 3, 4, 5, 6, 7, 8, 9, 10, 11, 12, 13, 14, 15, 16, 17, 18, 19, 20, 21, 22, 23, 24, 25, 26, 28, 29, 30, 31, 32, 33, 34, 35, 36, 37, 38, 39, 40, 41, 42, 43, 44, 45, 46, 47, or 48 weeks or more. For example, a compound having a formula (I), or a pharmaceutically acceptable salt, a stereoisomer, a diastereomer, an enantiomer or a racemate thereof (e.g., Compound I, or a pharmaceutically acceptable salt thereof) is administered six times per week for 1, 2, 3, 4, 5, 6, 7, 8, 9, 10, 11, 12, 13, 14, 15, 16, 17, 18, 19, 20, 21, 22, 23, 24, 25, 26, 28, 29, 30, 31, 32, 33, 34, 35, 36, 37, 38, 39, 40, 41, 42, 43, 44, 45, 46, 47, or 48 weeks or more. For example, 5 mg of a compound having a formula (I), or a pharmaceutically acceptable salt, a stereoisomer, a diastereomer, an enantiomer or a racemate thereof (e.g. Compound I, or a pharmaceutically acceptable salt thereof) is administered weekly (e.g. once per week, or two, three, four, five, or six times or more per week). For example, 10 mg of a compound having a formula (I), or a pharmaceutically acceptable salt, a stereoisomer, a diastereomer, an enantiomer or a racemate thereof (e.g. Compound I, or a pharmaceutically acceptable salt thereof) is administered weekly (e.g. once per week, or two, three, four, five, or six times or more per week). For example, 25 mg of a compound having a formula (I), or a pharmaceutically acceptable salt, a stereoisomer, a diastereomer, an enantiomer or a racemate thereof (e.g. Compound I, or a pharmaceutically acceptable salt thereof) is administered weekly (e.g. once per week, or two, three, four, five, or six times or more per week). For example, 50 mg of a compound having a formula (I), or a pharmaceutically acceptable salt, a stereoisomer, a diastereomer, an enantiomer or a racemate thereof (e.g. Compound I, or a pharmaceutically acceptable salt thereof) is administered weekly (e.g. once per week, or two, three, four, five, or six times or more per week). For example, 75 mg of a compound having a formula (I), or a pharmaceutically acceptable salt, a stereoisomer, a diastereomer, an enantiomer or a racemate thereof (e.g. Compound I, or a pharmaceutically acceptable salt thereof) is administered weekly (e.g. once per week, or two, three, four, five, or six times or more per week). For example, 100 mg of a compound having a formula (I), or a pharmaceutically acceptable salt, a stereoisomer, a diastereomer, an enantiomer or a racemate thereof (e.g. Compound I, or a pharmaceutically acceptable salt thereof) is administered weekly (e.g. once per week, or two, three, four, five, or six times or more per week).

### Pharmaceutical Compositions

In some embodiments, the disclosure provides compositions (e.g., pharmaceutical compositions) with desirable pharmacokinetic characteristics. For example, the compositions of the disclosure comprising a compound having a formula (I) or a pharmaceutically acceptable salt, a stereoisomer, a diastereomer, an enantiomer or a racemate thereof (e.g. Compound I, or a pharmaceutically acceptable salt thereof), may provide a blood level of the compound in its active form which, after metabolism e.g., to tenofovir (TFV) and/or tenofovir diphosphate (TFV-PP), results in blood levels of the metabolite that do not induce toxicity (e.g., nephrotoxicity). Similarly, the compositions may provide blood levels of an active form of a compound having a formula (I) or of a pharmaceutically acceptable salt, a stereoisomer, a diastereomer, an enantiomer or a racemate thereof (e.g. Compound I, or a pharmaceutically acceptable salt thereof), or TFV which are effective at treating HBV infection without inducing toxicity. Furthermore, the compositions may provide blood levels of an active form of a compound having a formula (I) or of a pharmaceutically acceptable salt, a stereoisomer, a diastereomer, an enantiomer or a racemate thereof (e.g. Compound I, or a pharmaceutically acceptable salt thereof), or TFV which remain detectable for 24 hours or more after administration.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

When the compounds of the present disclosure are administered as pharmaceuticals to mammals, e.g., humans, they can be given per se or as a pharmaceutical composition containing, for example, about 0.1% to about 99.9%, about 0.2 to about 98%, about 0.3% to about 97%, about 0.4% to about 96%, or about 0.5 to about 95% by weight of the active compounds (i.e., a compound having a formula (I) or a pharmaceutically acceptable salt, a stereoisomer, a diastereomer, an enantiomer or a racemate thereof, e.g. Compound I, or a pharmaceutically acceptable salt thereof) in combination with a pharmaceutically acceptable carrier. In some embodiments, a pharmaceutical composition containing about 0.5% to about 90% by weight of active ingredients in combination with a pharmaceutically acceptable carrier is suitable for administration to mammals, e.g., humans. Some embodiments provide preparation of a pharmaceutical composition comprising about 0.1% to about 99.9%, about 0.2 to about 98%, about 0.3% to about 97%, about 0.4% to about 96%, or about 0.5 to about 95% by weight of the active compounds for use in treating viral infections or viral infection associated disorders. The present disclosure provides use of about 0.1% to about 99.9%, about 0.2 to about 98%, about 0.3% to about 97%, about 0.4% to about 96%, or about 0.5 to about 95% by weight of the active compounds in the manufacture of a medicament containing effective amounts of the compound for use in treating viral infections and viral infection associated diseases.

The pharmaceutical compositions described herein can be given in unit dosage form. In some embodiments, the dosage unit form comprises about 5 mg of the compound (e.g., a compound having a formula (I) or a pharmaceutically acceptable salt, a stereoisomer, a diastereomer, an enantiomer or a racemate thereof, e.g. Compound I, or a pharmaceutically acceptable salt thereof). In some embodiments, the dosage unit form comprises about 10 mg of the compound (e.g., a compound having a formula (I) or a pharmaceutically acceptable salt, a stereoisomer, a diastereomer, an enantiomer or a racemate thereof, e.g. Compound I, or a pharmaceutically acceptable salt thereof). In some embodiments, the dosage unit form comprises about 15 mg or about 20 mg of the compound (e.g., a compound having a formula (I) or a pharmaceutically acceptable salt, a stereoisomer, a diastereomer, an enantiomer or a racemate thereof, e.g. Compound I, or a pharmaceutically acceptable salt thereof). In some embodiments, the dosage unit form comprises about 20 mg of the compound (e.g., a compound having a formula (I) or a pharmaceutically acceptable salt, a stereoisomer, a diastereomer, an enantiomer or a racemate thereof, e.g. Compound I, or a pharmaceutically acceptable salt thereof). In some embodiments, the dosage unit form comprises about 25 mg of the compound (e.g., a compound having a formula (I) or a pharmaceutically acceptable salt, a stereoisomer, a diastereomer, an enantiomer or a racemate thereof, e.g. Compound I, or a pharmaceutically acceptable salt thereof). In some embodiments, the dosage unit form comprises about 50 mg of the compound (e.g., a compound having a formula (I) or a pharmaceutically acceptable salt, a stereoisomer, a diastereomer, an enantiomer or a racemate thereof, e.g. Compound I, or a pharmaceutically acceptable salt thereof). In some embodiments, the dosage unit form comprises about 75 mg of the compound (e.g., a compound having a formula (I) or a pharmaceutically acceptable salt, a stereoisomer, a diastereomer, an enantiomer or a racemate thereof, e.g. Compound I, or a pharmaceutically acceptable salt thereof). In some embodiments, the dosage unit form comprises about 100 mg of the compound (e.g., a compound having a formula (I) or a pharmaceutically acceptable salt, a stereoisomer, a diastereomer, an enantiomer or a racemate thereof, e.g. Compound I, or a pharmaceutically acceptable salt thereof).

The active compounds described herein (i.e., a compound having a formula (I) or a pharmaceutically acceptable salt, a stereoisomer, a diastereomer, an enantiomer or a racemate thereof, e.g. Compound I, or a pharmaceutically acceptable salt thereof) may be combined with a pharmaceutically acceptable carrier according to conventional pharmaceutical compounding techniques.

In preparing compositions for oral dosage form any of the usual pharmaceutical media may be employed. Usual pharmaceutical media include, for example, water, glycols, oils, alcohols, flavoring agents, preservatives, coloring agents, and the like in the case of oral liquid preparations (such as for example, suspensions, solutions, emulsions and elixirs); aerosols; or carriers such as starches, sugars, microcrystalline cellulose, diluents, granulating agents, lubricants, binders, disintegrating agents and the like, in the case of oral solid preparations (such as for example, powders, capsules, and tablets).

Pharmaceutical compositions comprising the active compounds of the present disclosure may be formulated to have any concentration desired. In some embodiments, the composition is formulated such that it comprises at least a therapeutically effective amount.

In some embodiments, long-acting pharmaceutical compositions may be administered every 3 to 4 days, every week, or once every two weeks depending on half-life and clearance rate of the particular formulation.

The pharmaceutical compositions containing the active compound of the present disclosure (i.e., a compound having a formula (I) or a pharmaceutically acceptable salt, a stereoisomer, a diastereomer, an enantiomer or a racemate thereof, e.g. Compound I, or a pharmaceutically acceptable salt thereof) may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically.

Oral compositions generally include an inert diluent or an edible pharmaceutically acceptable carrier. They can be enclosed in gelatin capsules or compressed into tablets. For the purpose of oral therapeutic administration, the active compounds can be incorporated with excipients and used in the form of tablets, troches, or capsules. Oral compositions can also be prepared using a fluid carrier for use as a mouthwash, wherein the compounds in the fluid carrier are applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

The active compounds of the disclosure can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from, e.g., Alza Corporation and Nova Pharmaceuticals, Inc. Liposomal suspensions (including liposomes targeted to infected cells with monoclonal antibodies to viral antigens) can also be used as pharmaceutically acceptable carriers.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compounds calculated to produce the desired therapeutic effect in association with the required pharmaceutical carrier. The specification for the dosage unit forms of the disclosure are dictated by and directly dependent on the unique characteristics of the active compounds and the particular therapeutic effect to be achieved.

In some embodiments, the pharmaceutical composition is in a dosage unit form. In some embodiments, the dosage unit form comprises a compound having a formula (I) or a pharmaceutically acceptable salt, a stereoisomer, a diastereomer, an enantiomer or a racemate thereof (e.g. Compound I, or a pharmaceutically acceptable salt thereof) in an amount of 5-100 mg. For example, the dosage unit form comprises a compound having a formula (I) or a pharmaceutically acceptable salt, a stereoisomer, a diastereomer, an enantiomer or a racemate thereof (e.g. Compound I, or a pharmaceutically acceptable salt thereof) in an amount of 5, 10, 15, 20, 25, 30, 35, 40, 45, 50, 55, 60, 65, 70, 75, 80, 85, 90, 95 or 100 mg.

The formulations of the present disclosure are used in manufacturing a medicament for treatment of HBV.

The pharmaceutical compositions can be included in a container, pack, or dispenser together with instructions for administration.

The compounds of the present disclosure are capable of further forming salts. All of these forms are also contemplated within the scope of the claimed disclosure.

As used herein, “pharmaceutically acceptable salts” refer to derivatives of the compounds of the present disclosure (i.e., a compound having a formula (I) or a pharmaceutically acceptable salt, a stereoisomer, a diastereomer, an enantiomer or a racemate thereof, e.g. Compound I, or a pharmaceutically acceptable salt thereof) wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, diethylamine, diethylaminoethanol, ethylenediamine, imidazole, lysine, arginine, morpholine, 2-hydroxyethylmorpholine, dibenzylethylenediamine, trimethylamine, piperidine, pyrrolidine, benzylamine, tetramethylammonium hydroxide and the like.

It should be understood that all references to pharmaceutically acceptable salts include solvent addition forms (solvates) or crystal forms (polymorphs) as defined herein, of the same salt for a given compound).

The compounds of the present disclosure can also be prepared as esters, for example, pharmaceutically acceptable esters. For example, an alcohol group in a compound can be converted to its corresponding ester, e.g., acetate, propionate, or other esters.

Techniques for formulation and administration of the disclosed compounds of the disclosure can be found in Remington: the Science and Practice of Pharmacy, 19^{th} edition, Mack Publishing Co., Easton, Pa. (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

It is to be understood that while the disclosure has been described in conjunction with the preferred specific embodiments thereof, that the foregoing description as well as the examples that follow, are intended to illustrate and not limit the scope of the disclosure. It will be understood by those skilled in the art that various changes may be made and equivalents may be substituted without departing from the scope of the disclosure, and further that other aspects, advantages and modifications will be apparent to those skilled in the art to which the disclosure pertains.

All patents, patent applications, and publications mentioned herein are hereby incorporated by reference in their entireties. However, where a patent, patent application, or publication containing express definitions is incorporated by reference, those express definitions should be understood to apply to the incorporated patent, patent application, or publication in which they are found, and not to the remainder of the text of this application, in particular the claims of this application.

Other features and advantages of the present disclosure are apparent from the different examples. The provided examples illustrate different components and methodology useful in practicing the present invention. The examples do not limit the claimed invention. Based on the present disclosure the skilled artisan can identify and employ other components and methodology useful for practicing the present invention.

### EXAMPLES

### Example 1

### Synthesis of Compounds of the Disclosure

Exemplary methods for preparing compounds of the present disclosure are set forth below. In the following description, all variables are, unless otherwise noted, as defined in the formulas described herein. The following non-limiting descriptions illustrate the general methodologies that may be used to obtain the compounds described herein.

Compound I may be prepared in accordance with known procedures, or variations thereof that will be apparent to those skilled in the art. For example, Compound I may be synthesized using methods analogous to those previously described for 9-S-[3-hydroxy-2-(phosphonomethoxy) propyl]-adenine [(S)-HPMPA] derivatives (see Beadle et al., J. Med. Chem. 49, 2010-2015 (2006); Painter et al., Antimicrob. Agents Chemother. 51, 3505 (2007), and US Patent Application Publication No. 2007/0003516, each of which is incorporated herein by reference in its entirety).

In some embodiments, a salt of Compound I described herein may be prepared by dissolving Compound I in an appropriate solvent,

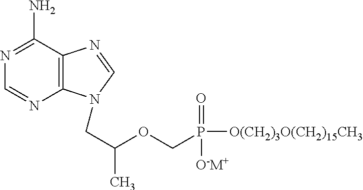

adding a suitable base to the mixture of the solvent and Compound I, and removing the solvent to provide the salts of Compound I.

The solvent used in the preparation may be any suitable solvent known to one skilled in the art or a combination of solvents that provides satisfactory yield of the product. In some embodiments, the solvent is a mixture of at least two solvents. Exemplary combination of solvents includes, but is not limited to, dichloromethane and methanol, dichloromethane and ethanol. In some embodiments, the molar ratio of the dichloromethane and methanol is in a range of about 1:1 to 9:1. In some embodiments, the molar ratio of the dichloromethane and methanol is in a range of about 7:3 to 9:1. In a further embodiment, the molar ratio of the dichloromethane and methanol is about 9:1.

The base used in the preparation may be any suitable base known to one skilled in the art or a combination of bases that provides satisfactory yield of the product. In some embodiments, the base is an alkali metal alcoholate base. Exemplary bases include, but are not limited to, potassium methoxide, sodium methoxide, lithium ter-butoxide, ammonium hydroxide, sodium hydroxide, potassium hydroxide, and lithium hydroxide.

The process described herein may further include the step of recrystallization to remove impurity, side products, and unreacted starting material. The recrystallization step comprises the step of dissolving the product in a suitable solvent at an appropriate temperature, cooling to an appropriate temperature for a sufficient period of time to precipitate the salts of Compound I, and filtering to provide the salts of Compound I. In some embodiments, the temperature for the step of dissolving is in a range of about 50° C. to 80° C.

### Example 2

### Phase I Clinical Trials with Healthy Adults

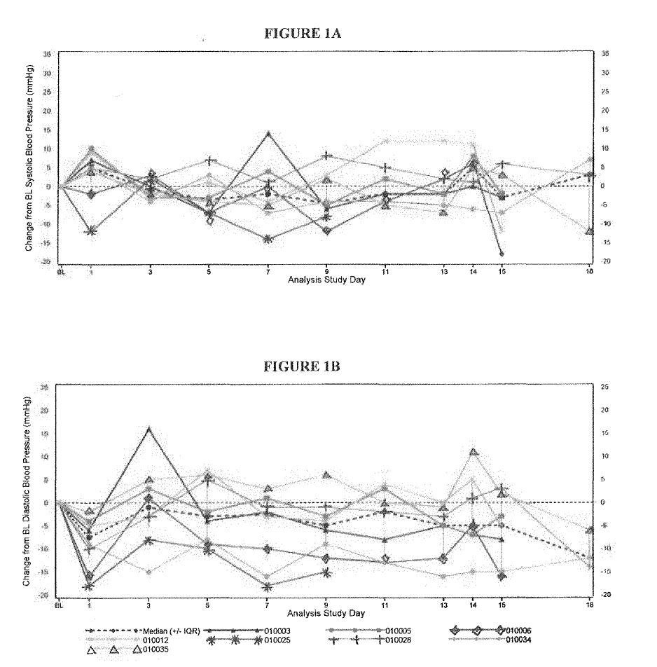

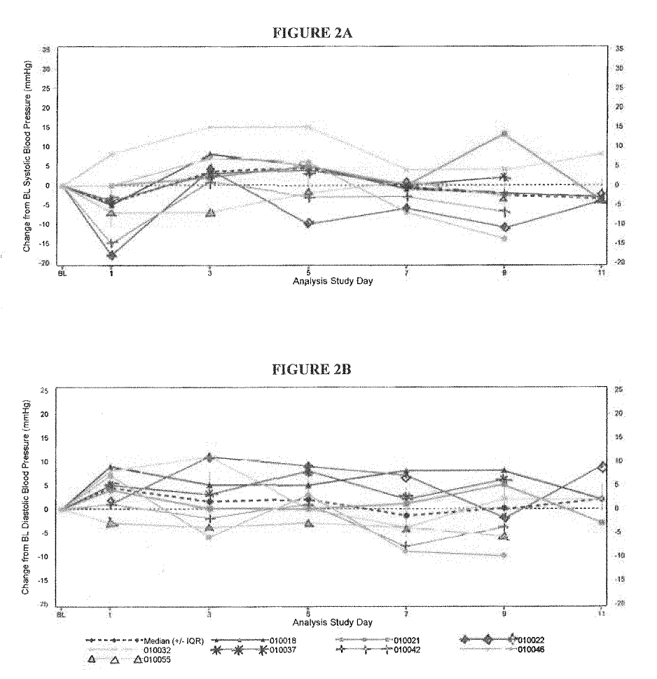

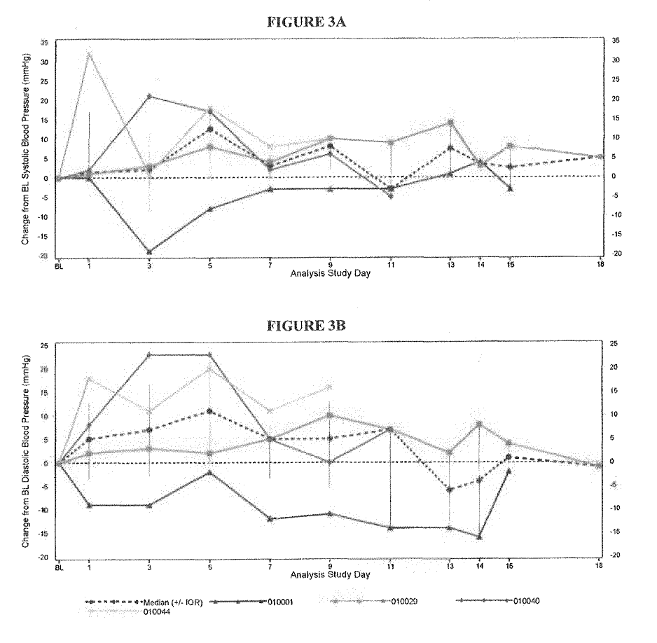

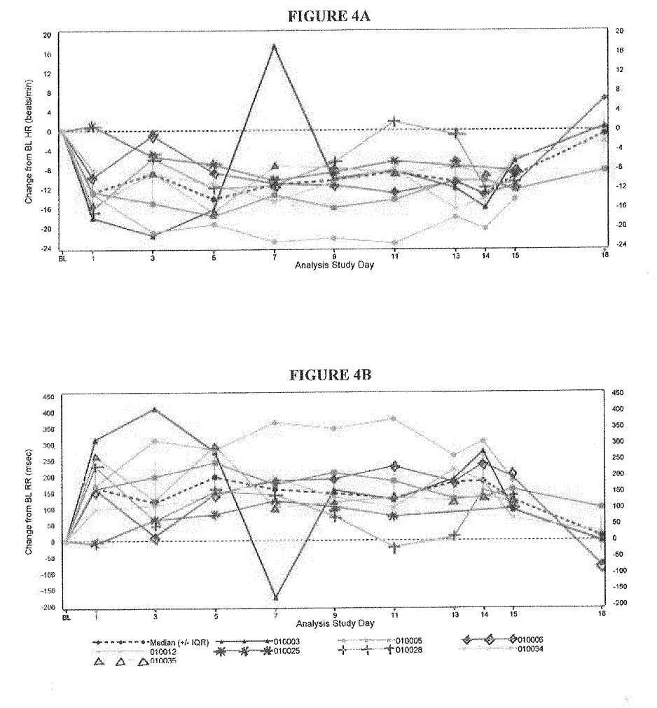

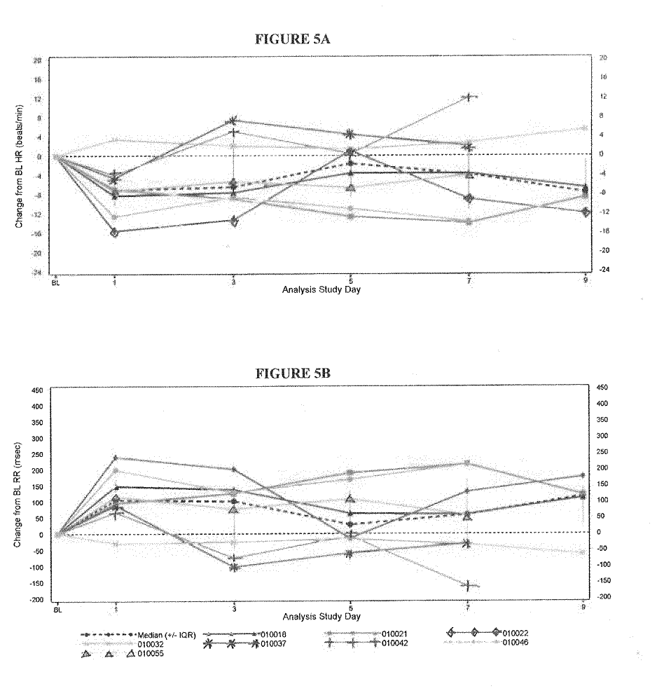

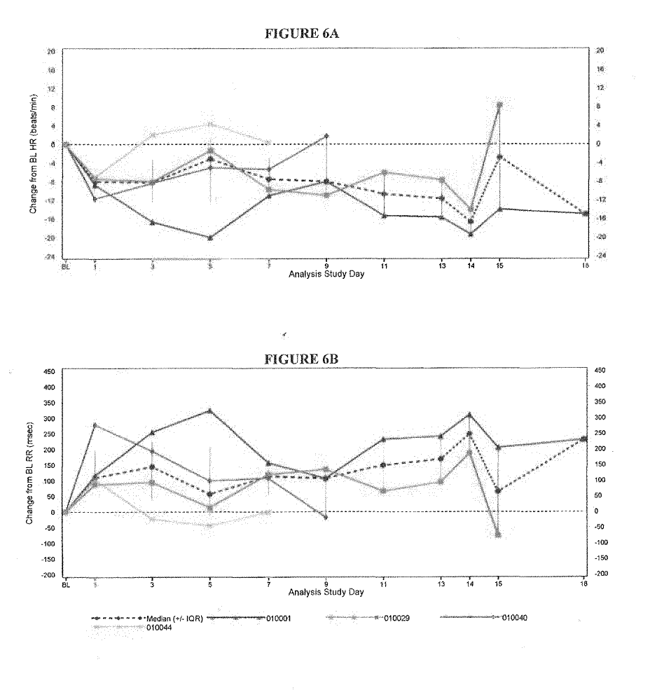

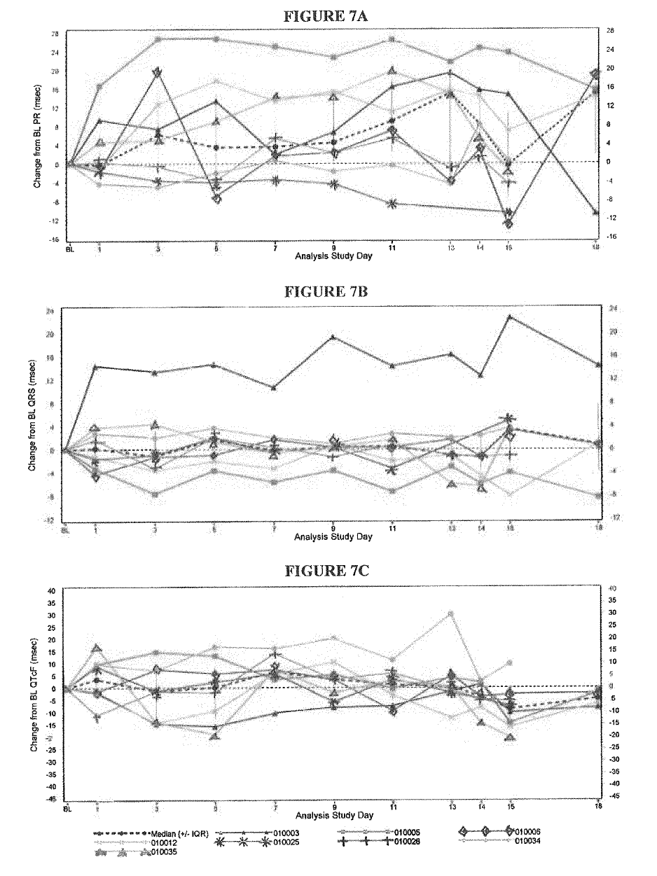

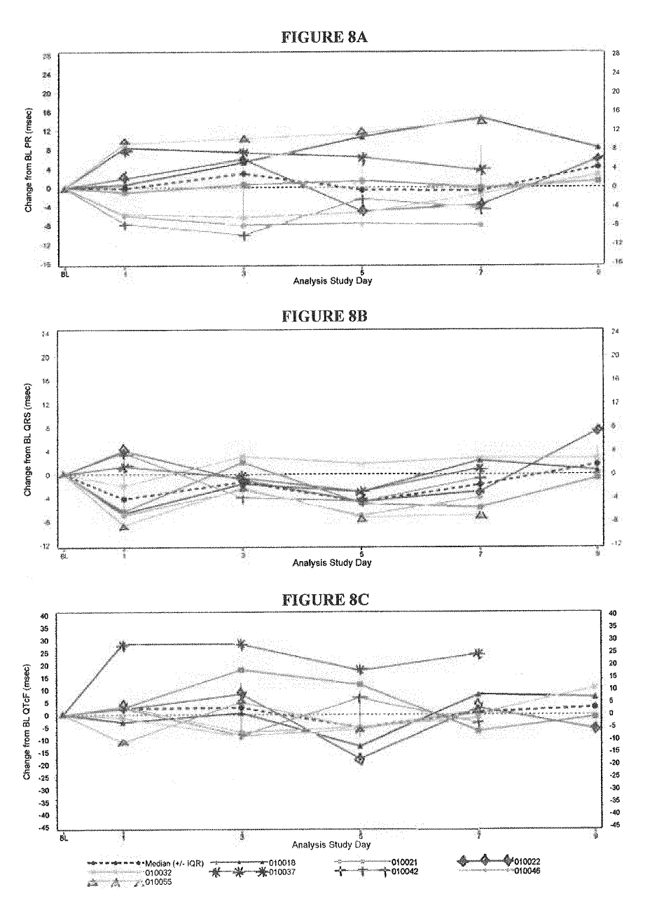

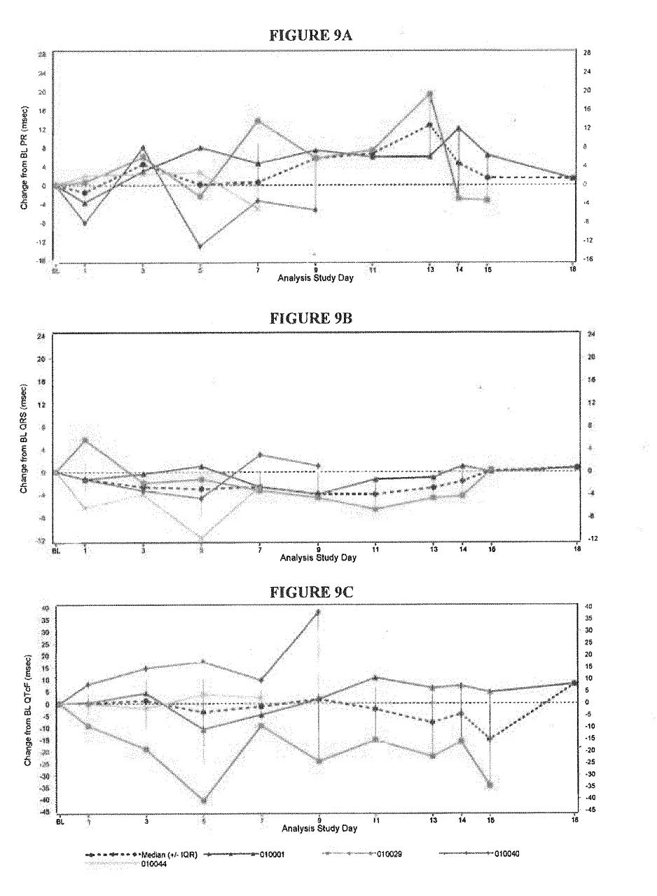

Eight healthy subjects were given Compound I orally at a daily dose of 5 mg for administration periods of between 9 and 14 days. Eight healthy subjects were given Compound I daily at a dose of 10 mg for administration periods of between 9 and 11 days. Additionally, a placebo was administered to four healthy subjects for administration periods of between 9 and 14 days. The subjects were evaluated for changes in baseline blood pressure (FIGS. 1A and 1B: Changes in systolic and diastolic blood pressure, respectively, after administration of 5 mg of Compound I; FIGS. 2A and 2B: Changes in systolic and diastolic blood pressure, respectively, after administration of 10 mg of Compound I; FIGS. 3A and 3B: Changes in systolic and diastolic blood pressure, respectively, after administration of placebo). Furthermore, electrocardiograms (ECGs) were recorded to evaluate the change in baseline of heart rate, RR intervals, PR intervals, QRS duration and QTcF in each subject (FIGS. 4A and 4B: Changes in heart rate and RR interval, respectively, after administration of 5 mg of Compound I; FIGS. 5A and 5B: Changes in heart rate and RR interval, respectively, after administration of 10 mg of Compound I; FIGS. 6A and 6B: Changes in heart rate and RR interval, respectively, after administration of placebo; FIGS. 7A, 7B, and 7C: Changes in PR interval, QRS duration, and QTcF duration, respectively, after administration of 5 mg of Compound I; FIGS. 8A, 8B and 8C: Changes in PR interval, QRS duration, and QTcF duration, respectively, after administration of 10 mg of Compound I; FIGS. 9A, 9B and 9C: Changes in PR interval, QRS duration, and QTcF duration, respectively, after administration of placebo). In the Figures, as well as in Table 2 below, individual subjects are identified by 6-digit numbers. Subjects that were administered 5 mg of Compound I, 10 mg of Compound I, or a placebo, were monitored for between 15 and 18 days, between 9 and 11 days, or between 9 and 18 days after initiation of the study, respectively. No subjects needed to discontinue the study prematurely. The baselines are defined as the last variable assessment prior to initiation of the study. The demographics and baseline characteristics for the tested population are presented in Table 1.

TABLE 1
Demographics and baseline characteristics - safety population

| | | | |
|---|---|---|---|
| Compound I | Compound I | Compound I | Placebo |
| 5 mg | 10 mg | Total | Total |
| (N = 8) | (N = 8) | (N = 16) | (N = 4) |

Age at Day 1 (years)

| n | 8 | | 8 | | 16 | | 4 | |
|---|---|---|---|---|---|---|---|---|
| Mean (SD) | 33.6 | (6.86) | 33.9 | (6.83) | 33.8 | (6.62) | 32.0 | (7.39) |

| Median | 33.0 | 32.5 | 32.5 | 33.0 |
|---|---|---|---|---|
| Min, Max | 25, 45 | 25, 44 | 25, 45 | 23, 39 |

Sex

| | | | | | | | | |
|---|---|---|---|---|---|---|---|---|
| Male | 5 | (62.5%) | 5 | (62.5%) | 10 | (62.5%) | 3 | (75.0%) |
| Female | 3 | (37.5%) | 3 | (37.5%) | 6 | (37.5%) | 1 | (25.0%) |

Race

| American Indian or | 0 | | 0 | | 0 | | 0 | |
|---|---|---|---|---|---|---|---|---|
| Alaska Native | | | | | | | | |
| Asian | 8 | (100.0%) | 8 | (100.0%) | 16 | (100.0%) | 4 | (100.0%) |

| | | | | |
|---|---|---|---|---|
| Black or African American | 0 | 0 | 0 | 0 |
| Native Hawaiian or | 0 | 0 | 0 | 0 |
| Other Pacific Islander | | | | |
| White | 0 | 0 | 0 | 0 |
| Other | 0 | 0 | 0 | 0 |

Ethnicity

| Hispanic or Latino | 0 | | 0 | | 0 | | 0 | |
|---|---|---|---|---|---|---|---|---|
| Not Hispanic or Latino | 8 | (100.0%) | 8 | (100.0%) | 16 | (100.0%) | 4 | (100.0%) |

Height (cm)

| n | 8 | | 8 | | 16 | | 4 | |
|---|---|---|---|---|---|---|---|---|
| Mean (SD) | 165.8 | (10.32) | 164.1 | (9.74) | 164.9 | (9.74) | 170.4 | (9.64) |

| Median | 167.5 | 163.5 | 165.0 | 170.0 |
|---|---|---|---|---|
| Min, Max | 151, 178 | 152, 181 | 151, 181 | 159, 183 |

Weight (kg)

| n | 8 | | 8 | | 16 | | 4 | |
|---|---|---|---|---|---|---|---|---|
| Mean (SD) | 62.8 | (11.94) | 63.4 | (9.19) | 63.1 | (10.29) | 65.9 | (15.53) |

| Median | 61.2 | 62.7 | 62.7 | 68.8 |
|---|---|---|---|---|
| Min, Max | 49, 80 | 51, 78 | 49, 80 | 46, 80 |

BMI (kg/m²)

| n | 8 | | 8 | | 16 | | 4 | |
|---|---|---|---|---|---|---|---|---|
| Mean (SD) | 22.6 | (1.95) | 23.5 | (2.16) | 23.1 | (2.04) | 22.5 | (3.61) |

| Median | 22.6 | 23.2 | 22.8 | 22.7 |
|---|---|---|---|---|
| Min, Max | 21, 26 | 21, 28 | 21, 28 | 18, 26 |

Few, minor adverse events (i.e., mild dizziness, headache or nausea), as well as few ECG abnormalities (i.e., intraventricular conduction defect, frequent ventricular complexes and ectopic supraventricular rhythm) were reported.

The pharmacokinetics of Compound I in human plasma was also evaluated. Plasma concentrations of Compound I and tenofovir (TFV) were measured at various time points up to 24 h after oral administration of a single 5 mg dose of Compound I. The results are summarized in Table 2.

TABLE 2
Pharmacokinetic plasma concentrations.

| Subject | Dose | Time Point | Compound I | TFV |
|---|---|---|---|---|
| 010003 | 5 mg | Pre-dose | <LLOQ < (0.150) | <LLOQ < (0.500) |

| | | | |
|---|---|---|---|
| 0.5 | hours post-dose | <LLOQ < (0.150) | <LLOQ < (0.500) |
| 1 | hour post-dose | <LLOQ < (0.150) | <LLOQ < (0.500) |
| 2 | hours post-dose | 0.180 | <LLOQ < (0.500) |
| 2.5 | hours post-dose | 1.070 | <LLOQ < (0.500) |
| 3 | hours post-dose | 1.690 | <LLOQ < (0.500) |
| 4 | hours post-dose | 2.460 | <LLOQ < (0.500) |
| 6 | hours post-dose | 0.891 | 0.560 |
| 10 | hours post-dose | <LLOQ < (0.150) | 0.526 |
| 12 | hours post-dose | <LLOQ < (0.150) | <LLOQ < (0.500) |
| 15 | hours post-dose | <LLOQ < (0.150) | <LLOQ < (0.500) |
| 18 | hours post-dose | <LLOQ < (0.150) | <LLOQ < (0.500) |
| 24 | hours post-dose | <LLOQ < (0.150) | <LLOQ < (0.500) |

| | | | | |
|---|---|---|---|---|
| 010005 | 5 mg | Pre-dose | <LLOQ < (0.150) | <LLOQ < (0.500) |

| | | | |
|---|---|---|---|
| 0.5 | hours post-dose | 0.303 | <LLOQ < (0.500) |
| 1 | hour post-dose | 1.310 | <LLOQ < (0.500) |
| 2 | hours post-dose | 2.030 | 0.519 |
| 2.5 | hours post-dose | 1.550 | 0.693 |
| 3 | hours post-dose | 1.260 | 0.764 |
| 4 | hours post-dose | 0.712 | 0.900 |
| 6 | hours post-dose | 0.179 | 0.908 |
| 10 | hours post-dose | <LLOQ < (0.150) | 0.695 |
| 12 | hours post-dose | <LLOQ < (0.150) | 0.535 |
| 15 | hours post-dose | <LLOQ < (0.150) | <LLOQ < (0.500) |
| 18 | hours post-dose | <LLOQ < (0.150) | <LLOQ < (0.500) |
| 24 | hours post-dose | <LLOQ < (0.150) | <LLOQ < (0.500) |

| | | | | |
|---|---|---|---|---|
| 010006 | 5 mg | Pre-dose | <LLOQ < (0.150) | <LLOQ < (0.500) |

| | | | |
|---|---|---|---|
| 0.5 | hours post-dose | 0.567 | <LLOQ < (0.500) |
| 1 | hour post-dose | 0.592 | <LLOQ < (0.500) |
| 2 | hours post-dose | 0.310 | <LLOQ < (0.500) |
| 2.5 | hours post-dose | 1.350 | 0.665 |
| 3 | hours post-dose | 1.440 | 0.900 |
| 4 | hours post-dose | 0.508 | 1.140 |
| 6 | hours post-dose | <LLOQ < (0.150) | 1.110 |
| 10 | hours post-dose | <LLOQ < (0.150) | 0.800 |
| 12 | hours post-dose | <LLOQ < (0.150) | 0.664 |
| 15 | hours post-dose | <LLOQ < (0.150) | 0.581 |
| 18 | hours post-dose | <LLOQ < (0.150) | <LLOQ < (0.500) |
| 24 | hours post-dose | <LLOQ < (0.150) | <LLOQ < (0.500) |

| | | | | |
|---|---|---|---|---|
| 010012 | 5 mg | Pre-dose | <LLOQ < (0.150) | <LLOQ < (0.500) |

| | | | |
|---|---|---|---|
| 0.5 | hours post-dose | <LLOQ < (0.150) | <LLOQ < (0.500) |
| 1 | hour post-dose | 2.980 | <LLOQ < (0.500) |
| 2 | hours post-dose | 8.530 | <LLOQ < (0.500) |
| 2.5 | hours post-dose | 5.250 | 0.708 |
| 3 | hours post-dose | 4.270 | 0.781 |
| 4 | hours post-dose | 1.500 | 0.948 |
| 6 | hours post-dose | 0.372 | 1.080 |
| 10 | hours post-dose | <LLOQ < (0.150) | 0.652 |
| 12 | hours post-dose | <LLOQ < (0.150) | <LLOQ < (0.500) |
| 15 | hours post-dose | <LLOQ < (0.150) | <LLOQ < (0.500) |
| 18 | hours post-dose | <LLOQ < (0.150) | <LLOQ < (0.500) |
| 24 | hours post-dose | <LLOQ < (0.150) | <LLOQ < (0.500) |

| | | | | |
|---|---|---|---|---|
| 010025 | 5 mg | Pre-dose | <LLOQ < (0.150) | <LLOQ < (0.500) |

| | | | |
|---|---|---|---|
| 0.5 | hours post-dose | 1.090 | <LLOQ < (0.500) |
| 1 | hour post-dose | 2.080 | <LLOQ < (0.500) |
| 2 | hours post-dose | 1.530 | 0.528 |
| 2.5 | hours post-dose | 1.340 | 0.596 |
| 3 | hours post-dose | 1.640 | 0.648 |
| 4 | hours post-dose | 0.520 | 0.750 |
| 6 | hours post-dose | <LLOQ < (0.150) | 0.711 |
| 10 | hours post-dose | <LLOQ < (0.150) | 0.516 |
| 12 | hours post-dose | <LLOQ < (0.150) | <LLOQ < (0.500) |
| 15 | hours post-dose | <LLOQ < (0.150) | <LLOQ < (0.500) |
| 18 | hours post-dose | <LLOQ < (0.150) | <LLOQ < (0.500) |
| 24 | hours post-dose | <LLOQ < (0.150) | <LLOQ < (0.500) |

| | | | | |
|---|---|---|---|---|
| 010028 | 5 mg | Pre-dose | <LLOQ < (0.150) | <LLOQ < (0.500) |

| | | | |
|---|---|---|---|
| 0.5 | hours post-dose | <LLOQ < (0.150) | <LLOQ < (0.500) |
| 1 | hour post-dose | <LLOQ < (0.150) | <LLOQ < (0.500) |
| 2 | hours post-dose | 0.753 | <LLOQ < (0.500) |
| 2.5 | hours post-dose | 1.500 | <LLOQ < (0.500) |
| 3 | hours post-dose | 4.430 | <LLOQ < (0.500) |
| 4 | hours post-dose | 1.540 | 0.594 |
| 6 | hours post-dose | 0.389 | 0.609 |
| 10 | hours post-dose | <LLOQ < (0.150) | 0.558 |
| 12 | hours post-dose | <LLOQ < (0.150) | <LLOQ < (0.500) |
| 15 | hours post-dose | <LLOQ < (0.150) | <LLOQ < (0.500) |
| 18 | hours post-dose | <LLOQ < (0.150) | <LLOQ < (0.500) |
| 24 | hours post-dose | <LLOQ < (0.150) | <LLOQ < (0.500) |

| | | | | |
|---|---|---|---|---|
| 010034 | 5 mg | Pre-dose | <LLOQ < (0.150) | <LLOQ < (0.500) |

| | | | |
|---|---|---|---|
| 0.5 | hours post-dose | 0.893 | <LLOQ < (0.500) |
| 1 | hour post-dose | 1.570 | <LLOQ < (0.500) |
| 2 | hours post-dose | 1.820 | 0.733 |
| 2.5 | hours post-dose | 0.206 | 0.959 |
| 3 | hours post-dose | 0.197 | 0.918 |
| 4 | hours post-dose | <LLOQ < (0.150) | 0.903 |
| 6 | hours post-dose | <LLOQ < (0.150) | 0.804 |
| 10 | hours post-dose | <LLOQ < (0.150) | 0.659 |
| 12 | hours post-dose | <LLOQ < (0.150) | 0.574 |
| 15 | hours post-dose | <LLOQ < (0.150) | <LLOQ < (0.500) |
| 18 | hours post-dose | <LLOQ < (0.150) | <LLOQ < (0.500) |
| 24 | hours post-dose | <LLOQ < (0.150) | <LLOQ < (0.500) |

| | | | | |
|---|---|---|---|---|
| 010035 | 5 mg | Pre-dose | <LLOQ < (0.150) | <LLOQ < (0.500) |

| | | | |
|---|---|---|---|
| 0.5 | hours post-dose | <LLOQ < (0.150) | <LLOQ < (0.500) |
| 1 | hour post-dose | 0.743 | <LLOQ < (0.500) |
| 2 | hours post-dose | 2.180 | <LLOQ < (0.500) |
| 2.5 | hours post-dose | 0.412 | 0.822 |
| 3 | hours post-dose | 2.080 | 0.891 |
| 4 | hours post-dose | 1.180 | 1.140 |
| 6 | hours post-dose | 0.448 | 1.370 |
| 10 | hours post-dose | <LLOQ < (0.150) | 1.300 |
| 12 | hours post-dose | <LLOQ < (0.150) | 1.140 |
| 15 | hours post-dose | <LLOQ < (0.150) | 1.040 |
| 18 | hours post-dose | <LLOQ < (0.150) | 0.759 |
| 24 | hours post-dose | <LLOQ < (0.150) | 0.599 |

LLOQ = Lower Limit of Quantification.

### Equivalents

The invention can be embodied in other specific forms without departing from the spirit or essential characteristics thereof. The foregoing embodiments are therefore to be considered in all respects illustrative rather than limiting on the invention described herein. Scope of the invention is thus indicated by the claims rather than by the foregoing description, and all changes that come within the meaning and range of equivalency of the claims are intended to be embraced therein.

1. A pharmaceutical composition comprising a compound having a formula: 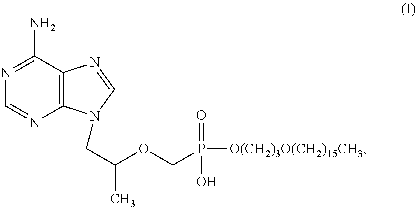
or a pharmaceutically acceptable salt, a stereoisomer, a diastereomer, an enantiomer or a racemate thereof in a dosage unit form comprising an amount from about 5 mg to about 100 mg of said compound, or pharmaceutically acceptable salt, stereoisomer, diastereomer, enantiomer or racemate thereof.

2. The pharmaceutical composition of claim 1, wherein the compound is: 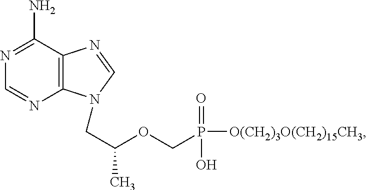
or a pharmaceutically acceptable salt thereof.

3. The pharmaceutical composition of claim 1, wherein the pharmaceutically acceptable salt of the compound is: 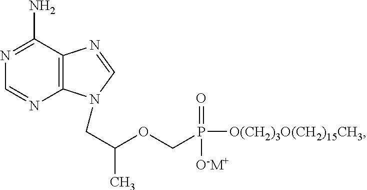
wherein M⁺ is Na⁺, Li⁺, K⁺, Ca²⁺, Mg²⁺, or NR_{c}R_{d}RₑR_{f}⁺, and R_{c}, R_{d}, Rₑ, and R_{f} are each independently hydrogen or C₁₋₅ alkyl, or a stereoisomer, a diastereomer, an enantiomer or a racemate thereof.

4. The pharmaceutical composition of claim 1, wherein the dosage unit form is in the form of an orally administered pill, capsule or tablet.

5. The pharmaceutical composition of claim 1, wherein the dosage unit form is in the form of an orally administered suspension, solution, emulsion, mouthwash or elixir.

6. The pharmaceutical composition of claim 1, wherein the dosage unit form comprises about 5 mg, about 10 mg, about 15 mg, about 20 mg, about 25 mg, about 50 mg, about 75 mg, or about 100 mg of the compound.

7. -13. (canceled)

14. A method for treating a viral infection in a subject, the method comprising administering to the subject a compound having a formula: 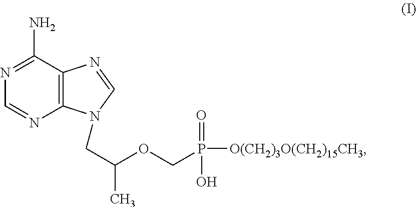
or a pharmaceutically acceptable salt, a stereoisomer, a diastereomer, an enantiomer or a racemate thereof, wherein said compound or pharmaceutically acceptable salt, stereoisomer, diastereomer, enantiomer or racemate thereof is administered orally to a subject at a dosage of about 5 mg to about 100 mg.

15. The method of claim 14, wherein the compound having a formula (I) is: 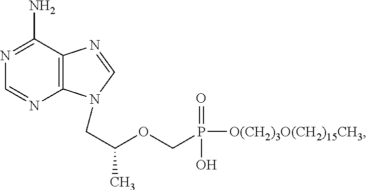
or a pharmaceutically acceptable salt thereof.

16. The method of claim 14, wherein the pharmaceutically acceptable salt of the compound having a formula (I) is: 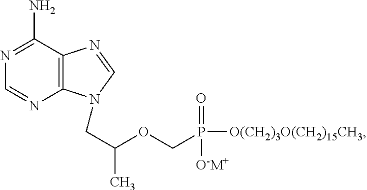
wherein M⁺ is Na⁺, Li⁺, K⁻, Ca²⁺, Mg²⁺, or NR_{c}R_{d}RₑR_{f}⁺, and R_{c}, R_{d}, Rₑ, and R_{f} are each independently hydrogen or C₁₋₅ alkyl, or a stereoisomer, a diastereomer, an enantiomer or a racemate thereof.

17. The method of claim 14, wherein the viral infection is an infection with a virus of the Hepadnaviridae family of viruses.

18. The method of claim 14, wherein the viral infection is an infection with hepatitis B virus.

19. The method of claim 14, wherein the compound is administered orally to a subject at a dose of about 5 mg, about 10 mg, about 15 mg, about 20 mg, about 25 mg, about 50 mg, about 75 mg, or about 100 mg.

20. -26. (canceled)

27. The method of claim 14, wherein said subject is a human being.

28. The method of claim 14, wherein the compound is administered daily.

29. The method of claim 28, wherein the compound is administered once per day for 1, 2, 3, 4, 5, 6, 7, 8, 9, 10, 11, 12, 13, 14, 15, 16, 17, 18, 19, 20, 21, 22, 23, 24, 25, 26, 27, or 28 days.

30. The method of claim 28, wherein the compound is administered two, three, or four or more times per day for 1, 2, 3, 4, 5, 6, 7, 8, 9, 10, 11, 12, 13, 14, 15, 16, 17, 18, 19, 20, 21, 22, 23, 24, 25, 26, 27, or 28 days.

31. The method of claim 14 wherein the compound is administered weekly.

32. The method of claim 31, wherein the compound is administered once per week for 1, 2, 3, 4, 5, 6, 7, 8, 9, 10, 11, 12, 13, 14, 15, 16, 17, 18, 19, 20, 21, 22, 23, 24, 25, 26, 28, 29, 30, 31, 32, 33, 34, 35, 36, 37, 38, 39, 40, 41, 42, 43, 44, 45, 46, 47, or 48 weeks.

33. The method of claim 31, wherein the compound is administered two, three, four, five or six or more times per week for 1, 2, 3, 4, 5, 6, 7, 8, 9, 10, 11, 12, 13, 14, 15, 16, 17, 18, 19, 20, 21, 22, 23, 24, 25, 26, 28, 29, 30, 31, 32, 33, 34, 35, 36, 37, 38, 39, 40, 41, 42, 43, 44, 45, 46, 47, or 48 weeks.

34. The method of claim 14, wherein the compound is administered for more than a month, more than six months, more than a year, more than five years, more than ten years, or for the duration of the subject's life.

35.-40. (canceled)
